(19) United States
(12) Patent Application Publication
(10) Pub. No.: US 2022/0047757 A1
(43) Pub. Date: Feb. 17, 2022

(54) Dual-Disinfection Germicial Lighting Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/002,038

(22) Filed: Aug. 25, 2020

Related U.S. Application Data
(63) Continuation-in-part of application No. 16/991,439, filed on Aug. 12, 2020.

(51) Int. Cl. A61L 9/16

(52) U.S. Cl. CPC A61L 9/16; A61L 2209/12; A61L 2209/21; A61L 2209/134; A61L 2209/16

(57) ABSTRACT

A dual-disinfecting germicidal lighting device includes a housing, an air-permeable porous carrier with at least two sides, a fan, two light sources, and two means of disinfection that are back-up means for each other. A first means of disinfection is an air-disinfection means and it includes the housing, the air-permeable porous carrier, the fan, and the first light source. The air-permeable porous carrier contains a photocatalyst material, and the first light source activates the photocatalyst material in air-permeable porous carrier. The housing, the air-permeable porous carrier, and the fan together form an air chamber. The fan forces the surrounding air through the air-permeable porous carrier. A second means of disinfection is an air-and-surface disinfection means that includes the second light source. The second light source is a germicidal light source that disinfects pathogens in the surrounding air or on a nearby surface through the shining of its light.

The present disclosure is part of a Continuation-in-Part (CIP) of U.S. patent application Ser. No. 16/991,439, filed 12 Aug. 2020, the content of which being incorporated by reference in its entirety.

### BACKGROUND

### Technical Field

### Description of Related Art

In U.S. patent application Ser. No. 16/991,439, an air-disinfecting photocatalytic device was introduced. The device comprises a housing, an air-permeable porous carrier with at least two sides, a fan, and a light source. The housing houses the air-permeable porous carrier, the fan, and the light source. The air-permeable porous carrier contains a photocatalyst material. The light source activates the photocatalyst material in the air-permeable porous carrier. The housing, the air-permeable porous carrier, and the fan together form an air chamber. The fan operates to either increase or deplete the air in the air chamber, resulting in an air pressure difference between a first air pressure inside the air chamber and a second air pressure outside the air chamber. As a result, the air pressure difference causes the air to pass through the air-permeable porous carrier from the high air pressure side of the air-permeable porous carrier to the low air pressure side of the air-permeable porous carrier. The air pressure in the air chamber may be higher or lower, depending on the airflow direction of the fan. As air passing through the air-permeable porous carrier, airborne pathogens are trapped on the surface of the air-permeable porous carrier. The photocatalyst material in the air-permeable porous carrier which has been activated by the light source would kill the pathogens trapped on the surface of the air-permeable porous carrier.

There are three limitations with the original patent application. Firstly, while the air-disinfecting photocatalytic device is effective against airborne pathogens and can be used continuously without any health concern, it has no effect against any pathogens on a surface. Some pathogens such as Escherichia. Coli (E. Coli), Staphylococcus Aureus, or MRSA, are transmitted mainly through physical contact when someone touch a surface contaminated with these pathogens. The invention introduced in U.S. patent application Ser. No. 16/991,439 is not useful for surface disinfection. Secondly, when the fan or the light source used the disclosure of U.S. patent application Ser. No. 16/991,439 failed, the air-disinfection function of the air-disinfecting photocatalytic device stops to work. Thirdly, when the air-permeable porous carrier is covered with dust and the activated photocatalyst material cannot make physical contact with airborne pathogens trapped on the surface of the carrier, the air-disinfection function of the device would become less effective.

The present disclosure proposes a dual-disinfection germicidal lighting device where it enhances the air-disinfecting photocatalytic device introduced in U.S. patent application Ser. No. 16/991,439 by incorporating a second air-and-surface germicidal disinfection means. The preset disclosure can thus overcome the three limitations mentioned above.

### SUMMARY

In one aspect, the dual-disinfecting germicidal lighting device comprises a housing, an air-permeable porous carrier with at least two sides, a fan, a first light source, a second light source, a first means of disinfection, and a second means of disinfection. The housing houses the air-permeable porous carrier, the fan, the first light source, and the second light source. The first means of disinfection is an air-disinfection means comprising the housing, the air-permeable porous carrier, the fan, and the first light source. The air-permeable porous carrier contains a photocatalyst material. The first light source activates the photocatalyst material in the air-permeable porous carrier. The housing, the air-permeable porous carrier, and the fan together form an air chamber. The fan operates to either increase or deplete the air in the air chamber, resulting in an air pressure difference between a first air pressure inside the air chamber and a second air pressure outside the air chamber. As a result, the air pressure difference causes the air to pass through the air-permeable porous carrier from the high air pressure side of the air-permeable porous carrier to the low air pressure side of the air-permeable porous carrier. The air pressure in the air chamber may be higher or lower, depending on the airflow direction of the fan. As air passing through air-permeable porous carrier, airborne pathogens are trapped on the surface of the air-permeable porous carrier. The photocatalyst material in the air-permeable porous carrier has been activated by the first light source and would kill the pathogens trapped on the surface of the air-permeable porous carrier.

The second means of disinfection is an air-and-surface disinfection means and it comprises the second light source. The light of the second light source emits out of the device. The second light source is a germicidal light source capable of disinfecting pathogens in the air or on a surface through the shining of its light. Moreover, the first means and the second means of disinfection are a backup air-disinfection means to each other. When the first means of disinfection stops to work due to the failure of the fan or the first light source, the second means of disinfection can continue to operate and provide air-disinfection through the shining of the light of the second light source. When the first means of disinfection becomes impeded due to the surface of the air-permeable porous carrier is covered by dust, the second means of disinfection can continue to operate and provide air-disinfection through the shining of the light of the second light source. On the other hand, when the second light source failed, the second means of disinfection stops to work. When this happens, the first means of disinfection can continue operate and provide air-disinfection. Moreover, when both the first means and the second means of disinfection are operating, they together achieve a more effective air-disinfection. This is because the fan will bring airborne pathogens closer to the device and they would be killed more effectively by the shining of the light of the second light source at a closer distant. Furthermore, the air, after being disinfected to some degree by the second light source, would contain less pathogens when passing through the air-permeable porous carrier, and the less pathogens could then be killed more effectively by the same amount of activated photocatalytic material on the air-permeable porous carrier.

In some embodiments, a main active ingredient of the photocatalyst material in the air-permeable porous carrier is titanium dioxide (TiO₂). In some other embodiments, the photocatalyst material further contains a secondary active ingredient comprising silver, gold, copper, zinc, nickel, or a combination thereof. These metals when embedded in a photocatalyst are known to enhance the photocatalytic activity with visible light. Alternatively, the metal photocatalyst material may be used without TiO₂ as the main active ingredient of the photocatalyst material. In such cases, titanium dioxide (TiO₂) is not used, and a main active ingredient of the photocatalyst material in the air-permeable porous carrier comprises silver, gold, copper, zinc, nickel, or a combination thereof.

In some embodiments, the first light source emits light mainly in the 200 nm to 400 nm wavelength range. These are UV light sources which are known to be effective in activating TiO₂ photocatalyst. In some other embodiments, the first light source emits light mainly in the 400 nm to 700 nm visible wavelength range. The visible light source may be used in conjunction with TiO₂ mixed a secondary active ingredient comprising silver, gold, copper, zinc, nickel, or a combination thereof.

Studies have shown the UVC wavelength centered around 265 nm has the best effect in killing bacteria and viruses. Most commercially available UVC light sources are centered around 254 nm wavelength due to the nature of their light source technology. It is also known that UVC wavelength could cause skin and eye damages to people exposed to UVC. One recent study by Columbia University affirms that far UVC light source at 222 nm is effective in killing bacteria and viruses, and yet it doesn't have the side effects of causing skin or eye damages (http://www.columbia.edu/˜djb3/papers/Germicidal%20Efficacy%20and%20Mammalian%20Skin%20Safety%20of%20222-nm%20UV%20Light/pdf). Moreover, a follow-up study shows the 222 nm far UVC light can efficiently and safely inactivates airborne human coronaviruses (https://www.nature.com/articles/s41598-020-67211-2). In some embodiments, the second light source emits the light with a wavelength in a range of 200 nm to 240 nm, i.e., in the far UVC range. In some other embodiments, the second light source emits the light with a wavelength in a range of 240 nm to 280 nm, which is the regular germicidal UVC range. In some other embodiments, the second light source emits the light with a wavelength in a range of 280 nm to 315 nm, which is the UVB range. In some other embodiments, the second light source emits the light with a wavelength in a range of 315 nm to 400 nm, which is the UVA range. In some other embodiments, the second light source emits the light with a wavelength in a range of 400 to 410 nm. It is foreseeable that a second light source may emit a light with a wavelength in a range from 200 nm to 410 nm or beyond.

In some embodiments, the first light source may reside in the air chamber, such that the first light source is positioned closer to the air-permeable porous carrier and may effectively activate the photocatalyst material in the air-permeable porous carrier. In some other embodiments, the first light source may reside outside the air chamber such that the first light source may be easily replaceable.

In some embodiments, the first light source and the air-permeable porous carrier are disposed in a way such that there is no obstruction in a line of sight between the first light source and the air-permeable porous carrier. Any obstruction in the line of sight between the first light source and the air-permeable porous carrier would reduce the total spectral power received by the air-permeable porous carrier from the first light source, thus reducing the photocatalytic activities of the photocatalyst material.

The effectiveness of the photocatalyst activity of the device depends on the physical contact of the airborne pathogens with the photocatalyst material in the air-permeable porous carrier. When the air-permeable porous carrier is covered with dusts, the photocatalytic killing effectiveness of the device against airborne pathogens will be reduced. Therefore, it is critical for the air-permeable porous carrier to be replaceable, and ideally without any tools. In some embodiments, the air-permeable porous carrier is replaceable by a user without using any tool.

A UV light source tends to have a shorter lifetime, as compared to, for example, the lifetime of the fan. In some embodiments, the first light source is replaceable by a user without using any tool. This is so that when the first light source expires, it can be easily replaced with a new one, thus extending the lifetime of the device. Similarly, in some embodiments, the second light source may be replaceable by a user without using any tool, for the germicidal light source is known to have a shorter lifetime. It is foreseeable that the second light source has a standard electric connector base, such as G13, G5, 2G11, G24Q, etc., and thus can be installed into the corresponding socket(s) on the housing without using any tool.

In some embodiments, the air-permeable porous carrier comprises non-woven fabric or melt-blown fabric, which is one of the most used air-permeable porous material. The TiO₂ photocatalyst material may be added to non-woven/melt-blown fabric through spraying a TiO₂ solution onto the fabric or through submerging the fabric in a TiO₂ solution. In some other embodiments, the air-permeable porous carrier comprises ceramic. In this case, the TiO₂ photocatalyst material may be added to the ceramic carrier through firstly submerging the carrier in a TiO₂ solution and followed by a heat-curing process. Alternatively, an evaporation process may be used to dope TiO₂ onto the ceramic carrier.

In some embodiments, a third light source may be used in the device. The housing houses the third light source, and the light of the third light source may emit out of the device. This third light source is a utility light source with a main purpose for illuminating the surrounding area of the device. With the addition of the third light source, the present disclosure may be used as a luminaire, on top of its dual-disinfection functionality. If the second light source emits UV wavelength, then it is foreseeable to have an embodiment of this device with two operation modes. The first mode is for general lighting where the third light source is on and the second light source is off. The second mode is for germicidal lighting where the third light source is off and the second light source is on. The first light source may be on during both operation modes for providing continuous air-disinfection.

In some embodiments, the third light source emits the light with a wavelength range >400 nm, i.e., in the visible wavelength range for general lighting. In some embodiments, the third light source may include light emitting diodes (LED).

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts a diagram of a dual-disinfecting germicidal lighting device with the first light source inside the air chamber.

FIG. 2 schematically depicts a diagram of a dual-disinfecting germicidal lighting device with the first light source outside the air chamber.

FIG. 3 schematically depicts an embodiment in the form of a linear troffer luminaire.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of germicidal lighting device having different form factors.

The present disclosure discloses a dual-disinfecting germicidal lighting device that has a housing, an air-permeable porous carrier with at least two sides, a fan, and two light sources and two means of disinfection. The first means of disinfection is an air-disinfection means that includes the housing, the air-permeable porous carrier, the fan, and the first light source. The second means of disinfection is an air-and-surface disinfection means that includes the second light source. The first means and the second means of disinfection are a backup air-disinfection means to each other.

### Example Implementations

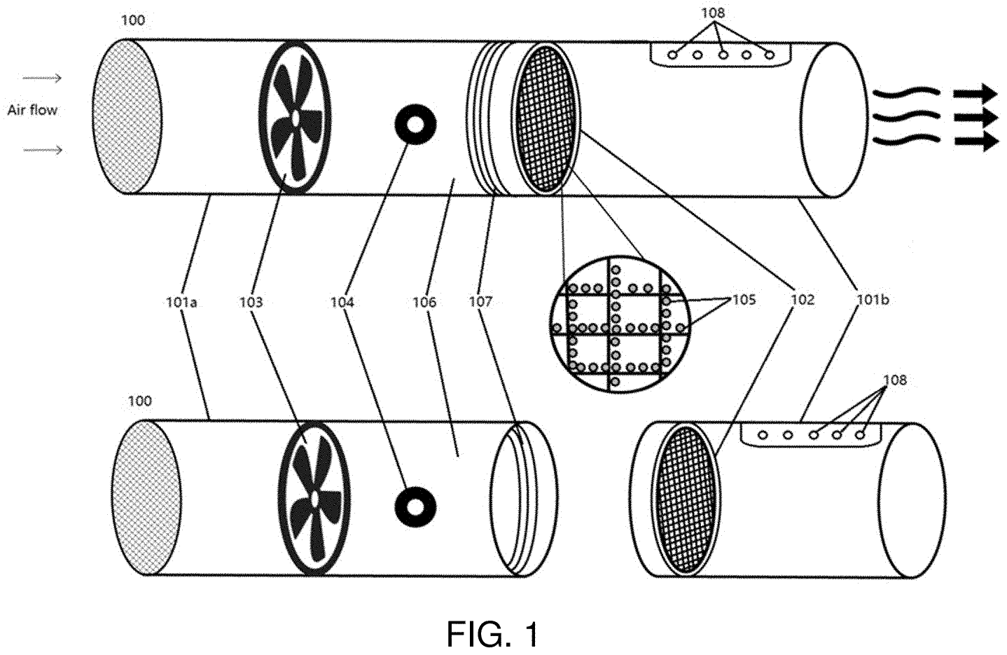

FIG. 1 is an embodiment of the dual-disinfecting germicidal lighting device of the present disclosure with a cylinder shape 100. The housing 101a, 101b houses the air-permeable porous carrier 102, the fan 103, the first light source 104, and the second light source 108. The air-permeable porous carrier 102 is made of ceramic and its surface is coated with a photocatalyst TiO₂ 105. The first means of disinfection is an air-disinfection means comprising the housing 101a, 101b, the air-permeable porous carrier 102, the fan 103, and the first light source 104. When the first light source 102 is on, it activates the photocatalyst 105. The housing 101a, 101b, and the air-permeable porous carrier 102, and the fan 103 together form an air chamber 106. The fan 103 operates to increase air pressure in the air chamber 106, forcing the air to exit out of the air chamber by passing from the left side of the air-permeable porous carrier 102 to the right side of the carrier. As the air passing through the air-permeable porous carrier 102, the airborne pathogens are trapped on the surface of the carrier, and the photocatalyst TiO₂ 105 being activated by the first light source 104 will kill the pathogens trapped on the surface of the carrier.

The first light source 104 emits light mainly in the 200 nm to 400 nm wavelength range. When a secondary active photocatalytic ingredient comprising silver, gold, copper, zinc, nickel, or a combination thereof is used in the photocatalyst 105, then the photocatalyst 105 may be activated by visible light. In which case, it is possible to use a visible light source emitting light mainly in the 400 nm to 700 nm wavelength range for the first light source 104. In this embodiment the first light source 104 is placed inside the air chamber 106. There is no obstruction in a line of sight between the first light source 104 and the air-permeable porous carrier 102.

The second means of disinfection is an air-and-surface disinfection means comprising the second light source 108. The second light source is housed on the surface of the housing 101b and emits its light out of the device. The wavelength range of the light from the second lighting source 108 may be in the 200-240 nm range, 240-280 nm range, 280-315 nm range, 315-400 nm range, 400-410 nm range, or a combination thereof.

During normal operation, both the first means and the second means of disinfection will operate simultaneously. When the first means of air-disinfection failed, the second means of disinfection provides continually air-disinfection with the shining its lighting into the surrounding air. When the second means of air-and-surface disinfection failed, the first means of air-disinfection continually disinfects the air.

The two sections of the housing, 101a and 101b, are connected through their threaded segment 107. These two sections of the housing 101a and 10ab can be disengaged by rotating the housing section 101b counterclockwise, without using any tool. Once the housing section 101b is disengaged from the housing section 101a, the air-permeable porous carrier 102 can be replaced with a new carrier. Similarly, the second light source 108 may be removed from the housing 101b for replacement without using any tool.

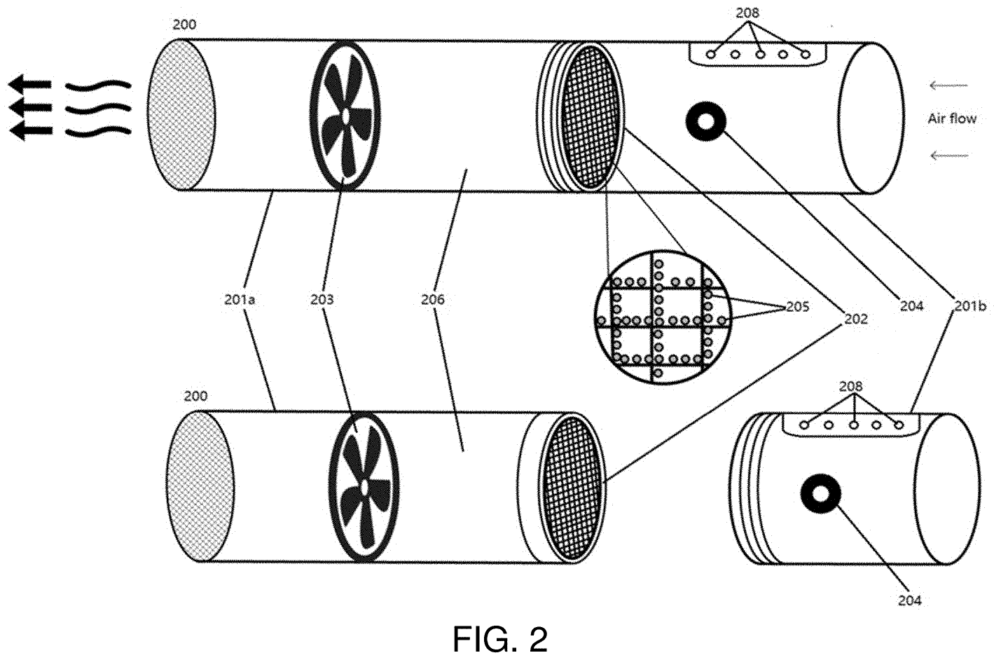

FIG. 2 is another embodiment of the dual-disinfection germicidal lighting device of the present disclosure with a cylinder shape 200. The housing 201a, 201b houses the air-permeable porous carrier 202, the fan 203, the first light source 204, and the second light source 208. The air-permeable porous carrier 202 is made of ceramic and its surface is coated with a photocatalyst TiO₂ 205. The first means of disinfection is an air-disinfection means comprising the housing 201a, 201b, the air-permeable porous carrier 202, the fan 203, and the first light source 204. The housing 201a, 201b, and the air-permeable porous carrier 202, and the fan 203 together form an air chamber 206. The fan 203 operates to deplete the air in the air chamber 206. As a result, the air pressure in the air chamber 206 will drop, forcing the air to pass from the right side of the air-permeable porous carrier 202 to the left side of the carrier and into the air chamber 206. As the air passing through the air-permeable porous carrier 202, the airborne pathogens are trapped on the surface of the carrier, and the photocatalyst TiO₂ 205 being activated by the first light source 204 will kill the pathogens trapped on the surface of the carrier.

The second means of disinfection is an air-and-surface disinfection means comprising the second light source 208. The second light source is housed on the surface of the housing 201b and emits its light out of the device. The wavelength range of the light from the second lighting source 108 may be in the 200-240 nm range, 240-280 nm range, 280-315 nm range, 315-400 nm range, 400-410 nm range, or a combination thereof.

In this embodiment the first light source 204 is placed outside the air chamber 206. There is no obstruction in a line of sight between the first light source 204 and the air-permeable porous carrier 202. The two sections of the housing, 201a and 201b, are connected through their threaded segment 207. These two sections of the housing 201a and 201b can be disengaged by rotating the housing section 201b counterclockwise, without using any tool. Once the housing section 201b is disengaged from the housing section 201a, the first light source 204 can be replaced with a new one. Similarly, the second light source 208 may be removed from the housing 201b for replacement without using any tool.

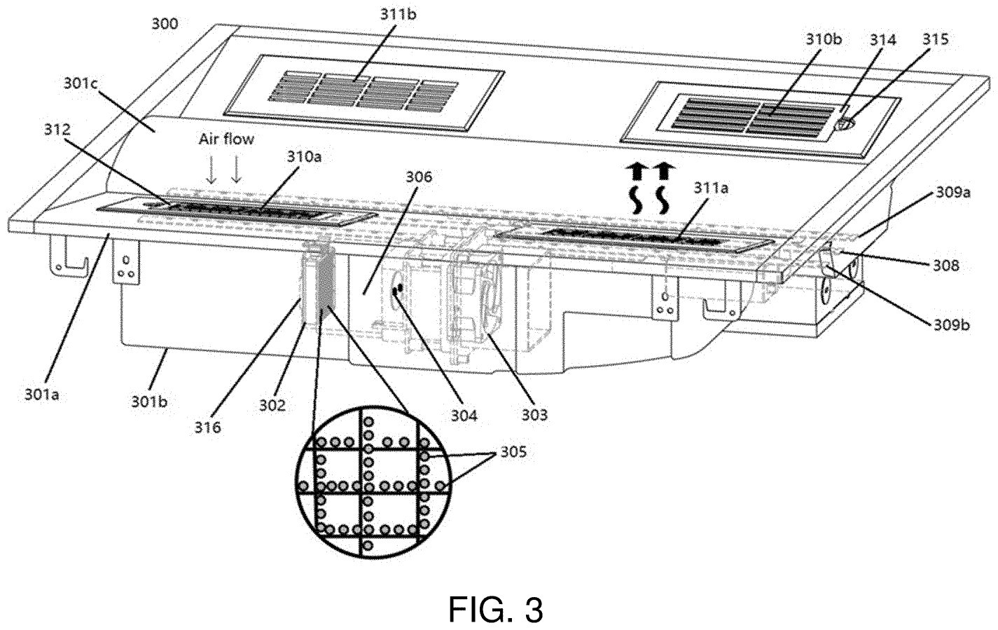

FIG. 3 shows an embodiment of the present disclosure in the form of a linear troffer luminaire 300. Behind the luminaire housing 301a, there are two air-processing modules, where the first air-processing module is shown in the front of the diagram and the second module, behind the first air-processing module, is not shown. Using the first air-processing module shown in the front as example, it includes an air inlet 310a, a ceramic air filter 302, a UVA LED assembly 304, a fan 303, an air outlet 311a, and a housing of the air-processing module 301b. The components of the two air-processing modules are the same. Therefore, with this embodiment, there are two ceramic air filters, two fans, and two UVA LED assemblies. In the center of the luminaire, there are three strips of LEDs: two of the strips 309a and 309b are visible light LED emitting for general lighting, and third strip 308 is a germicidal light source emitting a light in the UV wavelength for germicidal lighting operation.

The first means of disinfection is an air-disinfection means and it includes the housing 301a, 301b, the ceramic air filter 302, the fan 303, and the UVA LED assembly 304 as the first light source. The ceramic air filter 302 is coated with TiO₂ 305. The UVA LED assembly 304 emits a light to activate the photocatalyst TiO₂ 305. The housing 301a, 301b, the ceramic air filter 302, and the fan 303 together form an air chamber 306. The fan 303 sucks the air from its left so the air will pass from the left side of the ceramic air filter 302 to the right side of the filter into the air chamber 306. The airborne pathogens will be trapped on the surface of the ceramic air filter 302 and subsequently killed by the activated photocatalyst TiO₂ 305.

The second means of disinfection is an air-and-surface disinfection means and it includes the UV light source 309 as the second light source. The UV light source 309 is housed by the housing 301a and the lens cover 301c, and it emits UV light out of the device. The first and the second means of disinfection are backup air-disinfection means to each other. In this embodiment, the first light source 304 is placed inside the air chamber 306. There is no obstruction in a line of sight between the first light source 304 and the ceramic air filter 302.

The two strips of visible light LED 309a, 309b serve as the third light source of the device. The visible light LED 309a, 309b are housed by the housing 301a and the lens 301c, and they emit visible light with a wavelength in a range >400 nm out of the device for general lighting.

This embodiment may operate in two lighting modes: the general lighting and the germicidal lighting mode. During the general lighting mode, the third light source 309a, 309b is ON and the second light source 308 is OFF. During the germicidal lighting model, the second light source 308 is ON and the third light source 309a, 309b is OFF. With these two lighting modes, a user can switch this embodiment to the general lighting mode during office hours and then change it to the germicidal lighting mode in the evening for disinfecting the environment. With these two operation modes, the device avoids exposing a user to the UVC light emitted from the germicidal light source 308. Alternatively, a far UVC light source may be used for second light source 308, and in which case, the second light source 308 can be ON at all time for disinfecting the environment without the side effect of causing skin or eye damages to a user.

On the air inlet 310a, there is a removeable cover 312. Similarly, on the air inlet 310b, there is a removeable cover 314. There is latch 315 on one side of the cover 314. A user can push the latch 315 to lift and remove the cover 314 (similarly for removing the cover 312). Once the cover 314 or 312 is removed, the user can reach in and pull the ceramic air filter 302 out of its sitting slot 316 for replacement. A new ceramic air filter 302 can be slid into the sitting slot 316. This air filter replacement process can be done without using any tool.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A dual-disinfecting germicidal lighting device, comprising
a housing;
an air-permeable porous carrier with at least two sides;
a fan;
a first light source;
a second light source;
a first means of disinfection;
a second means of disinfection;
wherein:
the housing houses the air-permeable porous carrier, the fan, the first light source, and the second light source,
the first means of disinfection is an air-disinfection means comprising the housing, the air-permeable porous carrier, the fan, and the first light source, wherein:
the air-permeable porous carrier contains a photocatalyst material,
the first light source emits a first light to activate the photocatalyst material in the air-permeable porous carrier,
the housing, the air-permeable porous carrier, and the fan together form an air chamber,
the fan operates to either increase or deplete an amount of air in the air chamber, resulting in an air pressure difference between a first air pressure inside the air chamber and a second air pressure outside the air chamber, thereby causing the air to pass through the air-permeable porous carrier from a high air pressure side of the air-permeable porous carrier to a low air pressure side of the air-permeable porous carrier,
airborne pathogens are trapped on a surface of the air-permeable porous carrier when the air passes through the air-permeable porous carrier,
the photocatalyst material in the air-permeable porous carrier being activated by the first light source kills the pathogens trapped on the surface of the air-permeable porous carrier,
the second means of disinfection is an air-and-surface disinfection means comprising the second light source, wherein:
the second light source emits a second light out of the device,
the second light source is a germicidal light source capable of disinfecting pathogens in the air or on a surface through shining of the second light, and
the first and the second means of disinfection are a backup air-disinfection means to each other.

2. The dual-disinfecting germicidal lighting device of claim 1, wherein a main active ingredient of the photocatalyst material in the air-permeable porous carrier is titanium dioxide (TiO₂).

3. The dual-disinfecting germicidal lighting device of claim 2, wherein the photocatalyst material contains a secondary active ingredient comprising silver, gold, copper, zinc, nickel, or a combination thereof.

4. The dual-disinfecting germicidal lighting device of claim 1, wherein a main active ingredient of the photocatalyst material in the air-permeable porous carrier comprises silver, gold, copper, zinc, nickel, or a combination thereof.

5. The dual-disinfecting germicidal lighting device of claim 1, wherein the first light source emits the first light with a wavelength in a range of 200 nm to 400 nm.

6. The dual-disinfecting germicidal lighting device of claim 1, wherein the first light source emits the first light with a wavelength in a range of 400 nm to 700 nm.

7. The dual-disinfecting germicidal lighting device of claim 1, wherein the second light source emits the second light with a wavelength in a range of 200 nm to 240 nm.

8. The dual-disinfecting germicidal lighting device of claim 1, wherein the second light source emits the second light with a wavelength in a range of 240 nm to 280 nm.

9. The dual-disinfecting germicidal lighting device of claim 1, wherein the second light source emits the second light with a wavelength in a range of 280 nm to 315 nm.

10. The dual-disinfecting germicidal lighting device of claim 1, wherein the second light source emits the second light with a wavelength in a range of 315 to 400 nm.

11. The dual-disinfecting germicidal lighting device of claim 1, wherein the second light source emits the second light with a wavelength in a range of 400 to 410 nm.

12. The dual-disinfecting germicidal lighting device of claim 1, wherein the first light source resides in the air chamber.

13. The dual-disinfecting germicidal lighting device of claim 1, wherein the first light source resides outside the air chamber.

14. The dual-disinfecting germicidal lighting device of claim 1, wherein the first light source and the air-permeable porous carrier are disposed in a way such that there is no obstruction in a line of sight between the first light source and the air-permeable porous carrier.

15. The dual-disinfecting germicidal lighting device of claim 1, wherein the air-permeable porous carrier is replaceable without using any tool.

16. The dual-disinfecting germicidal lighting device of claim 1, wherein the first light source is replaceable without using any tool.

17. The dual-disinfecting germicidal lighting device of claim 1, wherein the second light source is replaceable without using any tool.

18. The dual-disinfecting germicidal lighting device of claim 1, wherein the air-permeable porous carrier comprises non-woven fabric or melt-blown fabric.

19. The dual-disinfecting germicidal lighting device of claim 1, wherein the air-permeable porous carrier comprises ceramic.

20. The dual-disinfecting germicidal lighting device of claim 1, further comprising a third light source, wherein:
the housing houses the third light source,
the third light source emits a third light out of the device, and
the third light source is a utility light source that illuminates a surrounding area of the device.

21. The dual-disinfecting germicidal lighting device of claim 19, wherein the third light source emits the third light with a wavelength greater than 400 nm.

22. The dual-disinfecting germicidal lighting device of claim 19, wherein the third light source comprises one or more light emitting diodes (LEDs).
